(19) United States
(12) Patent Application Publication
(10) Pub. No.: US 2014/0127309 A1
(43) Pub. Date: May 8, 2014

(54) ENCAPSULATED POLAR MATERIALS AND METHODS OF PREPARATION

(71) Applicants: Ian J. Drake, Philadelphia, PA (US); Andrew Hughes, Richboro, PA (US); Christopher J. Tucker, Midland, MI (US); Thomas H. Kalantar, Midland, MI (US); Joshua S Katz, Merion Station, PA (US)

(72) Inventors: Ian J. Drake, Philadelphia, PA (US); Andrew Hughes, Richboro, PA (US); Christopher J. Tucker, Midland, MI (US); Thomas H. Kalantar, Midland, MI (US); Joshua S Katz, Merion Station, PA (US)

(73) Assignees: ROHM and HAAS COMPANY, Philadelphia, PA (US); Dow Global Technologies LLC, Midland, MI (US)

(21) Appl. No.: 14/122,437

(22) PCT Filed: May 31, 2012
(86) PCT No.: PCT/US12/40158

§ 371 Date: Nov. 26, 2013

Related U.S. Application Data
(60) Provisional application No. 61/493,070, filed on Jun. 3, 2011.

(51) Int. Cl. B01J 13/16; C12N 11/04; B01J 13/18; A61K 9/50

(52) U.S. Cl. CPC B01J 13/16; A61K 9/5031; C12N 11/04; B01J 13/185
USPC 424/497; 502/159; 524/507; 524/850

(57) ABSTRACT

The present invention meets one or more of the above needs and is a composition comprising plurality of capsules wherein the capsules comprise: a core of one or more highly polar liquids; one or more polar active materials dissolved in or dispersed in one or more highly polar liquids; a mixture of one or more polymers and one of more highly polar liquids; or a mixture of one or more polymers, one or more highly polar liquids and one or more polar active materials, and a shell comprising, particles in a polymer matrix or particles; wherein the thickness of the shell is sufficient to prevent passage of the highly polar liquid or the active material through the shell or to control the rate passage of the highly polar liquid or the active material through the shell with the proviso that the one or more polymers may be located in the core, in the polymer matrix of the shell or both.

### CLAIM OF PRIORITY

This application claims priority from provisional application Ser. No. 61/493,070 filed Jun. 3, 2011 incorporated herein by reference in its entirety.

### FIELD OF THE INVENTION

The present invention relates to capsules containing polar materials in a core, particles in a shell and a polymer in one or both of the shell and the core, methods of preparation of capsules and the use of such capsules.

### BACKGROUND OF INVENTION

In many systems that utilize active chemical ingredients to perform a function, there is a need to control the contact of one or more active chemical ingredients with other components of the system. The active chemical ingredients react upon contact and to insure the ingredients react at the proper time they must be kept separate until the desired reaction is needed. A common approach is to set the system up as a two or more part system wherein the reactive ingredients are contacted just before use, examples include two part epoxy and polyurethane systems. Is some systems two part delivery systems are too complex, bulky or do not accommodate the scale of the reactive system, for example carbonless paper systems and adhesive systems. In other reactive systems, the active ingredient acts on an environment external to the reactive system, for instance humans, animals, plants or pests. In these systems either the active ingredient must be contacted with the environment at a precise time or gradually released to the environment, examples include drugs, agricultural chemicals, insecticides and the like. In some systems the size of the delivery system has to be very small, on the nanometer or micron scale to be effective, for example carbonless paper, drugs and agricultural chemicals. Encapsulation generally involves dispersing or dissolving an active ingredient in a polar or non-polar solvent and forming an emulsion or suspension with an incompatible solvent, either nonpolar or polar respectively. The component with the active ingredient is preferably in the discontinuous phase and forms discrete droplets in the continuous phase. Polymer forming components are either included in the two phases or added after formation of the emulsion or suspension. After formation of the droplets containing the active ingredient, a polymer is formed at the surface of the droplets, See Jahns et al. U.S. Pat. No. 7,572,397; Wulff et al. U.S. Pat. No. 6,890,653; and Kawai et al. U.S. Pat. No. 7,147,915, incorporated herein by reference. The polymer can fee formed by interfacial polymerization, in-situ polymerization, electrostatic deposition as a result of coacervation, precipitation and the like. The resulting structure is a particle having a core shell morphology, with a core of the active ingredient in a solvent or dispersant and a shell of a polymer.

Conventional methods for encapsulation, creating small particles with core-shell morphologies, require specific tuning of processing conditions and chemistry, and include seed-swell, high shear homogenization and sonication. Synthetic approaches to shell formation can include electrostatic deposition (layer-by-layer and coacervation), interfacial polymerization (polycondensation), deposition precipitation (urea-formaldehyde, melamine-femaldehyde, etc.) and free radical polymerization. While the synthetic approach is designed to lock the active ingredient in the particle following shell formation, particle creation techniques (such as seed-swell, high shear or sonication) can impact the extent of encapsulation. Conventional techniques known in the literature often require surfactants to reduce interfacial tension to aid particle creation. If the critical micelle concentration is exceeded, active ingredients with low water solubility can partially partition to the micelle during particle creation and shell formation, and a fraction of the active molecule can remain exterior to the encapsulated particle. An encapsulation technique that avoids the use of conventional surfactants would be an advantage. A technique known as “Pickering” emulsion stabilization can be used to stabilize discontinuous phases in emulsions or suspensions that do not require conventional surfactants. This technique uses small solid particles to reduce the interfacial tension at the oil/water interface.

Conventional encapsulation techniques exhibit other drawbacks. One drawback is that the active ingredient may migrate through the shell to contact the environment in which the encapsulated particles are contained. This is a problem where the particles are small and the shell is very thin. The shells of such small particles can also break with the application of slight pressure. This is a problem where the system is subjected to handling or pressures before intended use, for instance carbonless paper, agricultural chemicals or cure on demand adhesives. Pickering emulsions have been used in oil in water (non-polar solvent in polar solvent) systems to address those problems, see Jahns et al. U.S. Pat. No. 7,572397; Kawai et al. U.S. Pat. No. 7,147,915. These systems work well with relatively hydrophobic (nonpolar) active materials but do not work well with relatively hydrophilic (polar) active materials. McElroy et al. Macromolecules 2010, 43, 1855-1859, “Microencapsulation of a Reactive Liquid-Phase Amine for Self-Healing Epoxy Composites” discloses encapsulating relatively hydrophobic amines in nonpolar liquid.

There is a need for stable encapsulated particles having a core shell structure that contains relatively polar active materials that exhibit relatively high shell strength wherein the shell has controlled active agent permeability. There is a need for processes to prepare particles that facilitate control of the particle sizes, and for particles that do not contain surfactants.

### SUMMARY OF THE INVENTION

The present invention is a composition comprising a plurality of capsules wherein the capsules comprise: a core of one or more highly polar liquids, one or more polar active materials dissolved in or dispersed in one or more highly polar liquids, a mixture of one or more polymers and one or more highly polar liquids, or a mixture of one or more polymers, one or more highly polar liquids and one or more polar active materials; and a shell comprising particles in a polymer matrix or particles; wherein the thickness of the shell is sufficient to prevent passage of the highly polar liquid or the active material through the shell or to control the rate passage of the highly polar liquid or the active material through the shell with the proviso that the one or more polymers may be located in the core, in the polymer matrix of the shell or both. Preferably the diameter of the capsules is of a size suitable for encapsulating an active ingredient for the desired use. The core may comprise one or more highly polar liquids or one or more polar active materials dissolved in or dispersed in one or more highly polar liquids; and the shell comprises particles in a polymer matrix. In embodiments where the core comprises a mixture of one or more polymers and one or more highly polar liquids; or a mixture of one or more polymers, one or more highly polar liquids and one or more polar active materials; and the shell may comprise particles. The core may comprise one or more highly polar liquids. The core may comprise one or more active materials dissolved or dispersed in one or more highly polar liquids. Preferably the particles are solid particles that have a surface energy that promotes their migration to the interface of an emulsion or suspension of a highly polar liquid in a nonpolar liquid. The capsules may exhibit a size of about 50 nanometers or greater. The capsules may exhibit a size of about 500,000 nanometers or less.

In another aspect the invention is a process comprising: a) contacting a dispersion of particles in one or more non-polar liquids with one or more highly polar liquids wherein the particles have a surface energy that promotes migration to the interface of the emulsion or suspension of the highly polar liquids in the nonpolar liquids; b) emulsifying the contacted liquids to form an emulsion or suspension of the highly polar liquids in the non-polar liquids wherein discrete droplets of the highly polar liquid are formed having a portion of the particles on the surface of the droplets of highly polar liquid; and c) forming a polymer which forms a polymeric shell about the droplets of highly polar liquid wherein the polymeric shells comprise a portion of the particles; forms a mixture of the polymer and the highly polar liquid, and optionally the active material, in the core; or forms both. In one preferred embodiment, step c) comprises forming polymeric shells about the droplets of highly polar liquid wherein the polymeric shells comprise a portion of the particles. The highly polar liquid may be a polymer forming component. The highly polar liquid may contain one or more polymer forming components and one or more active materials. The highly polar liquid may contain one or more active materials. The polymer may be formed by any known process for preparing a polymer that facilitates depositing the polymer on the droplets at the interface of the nonpolar liquid and the highly polar liquid, for example interfacial polymerization, in-situ polymerization, precipitation of the polymer from the nonpolar or polar phase, anionic polymerization and electrostatic deposition, such as by coacervation or layer-by layer deposition. In interfacial polymerization the polymer forming components preferably comprise one or more relatively non-polar polymer forming components located in the nonpolar phase and one or more polar polymer forming component in the polar phase.

The capsules of the invention exhibit sufficient strength to prevent premature capsule rupture, facilitate the encapsulation of relatively polar active materials and do not contain residual surfactant. The capsules can be designed to have desired permeability of active components therethrough to prevent release without capsule rupture or to control the release of active ingredients. By the choice of particles contained in the shell, a controlled surface charge can be placed on the shell surface. The size of the capsules can be controlled by the choice and amount of the particles. The capsules of the invention can be used in any composition that utilizes relatively polar (hydrophilic) active components including liquid crystals, bioactive small molecules (biocides, insecticides, herbicides, etc.), fragrances, drugs, dyes/pigments, coalescing agents, reactive intermediates (hardeners, accelerators and catalysts for epoxy and other 2K reactive systems), photoactive agents, flavorings, fertilizers, cosmetic active ingredients, DNA, RNA, proteins, cellular material, sugars, cells (for example red blood cells, white blood cells), and the like. The capsules can be used in dry-film protection, marine anti-fouling, oil/gas treatment, agricultural treatments, drug delivery, catalysts, selective absorption (chromatography), water treatment, cure-on-demand polymerization, personal care, and corrosion, resistance. One skilled in the art upon learning of the invention of this patent application would recognize other materials that could be encapsulated in the capsules of the invention. The capsules of active materials can be used in the manner known to those skilled in the art.

### DESCRIPTION OF THE DRAWINGS

FIG. 1 is a drawing depicting one embodiment of the capsules of the invention.

FIG. 2 is a drawing depicting a second embodiment of the capsules of the invention.

FIG. 3 is a drawing depicting a third embodiment of the capsules of the invention.

FIG. 4 shows an optical micrograph of capsules formed.

### DETAILED DESCRIPTION

The explanations and illustrations presented herein are intended to acquaint others skilled in the art with the invention, its principles, and its practical application. Those skilled in the art may adapt and apply the invention in its numerous forms, as may be best suited to the requirements of a particular use. The specific embodiments of the present invention, as set forth are not intended as being exhaustive or limiting of the invention. The scope of the invention should be determined not with reference to the above description, but should be determined with reference to the appended claims, along with the full scope of equivalents to which such claims are entitled. The disclosures of all articles and references, including patent applications and publications, are incorporated by reference for all purposes. Other combinations are possible as will be gleaned from the following claims, which are hereby incorporated by reference herein.

The invention relates to encapsulated capsules having a shell containing solid particles and core containing a polar active material and to processes for their preparation and relates to compositions utilizing such capsules and processes for using such particles. Polar as used herein means a compound that contains bonds having a significant difference in electronegativity, there is a separation of the electronic charge in the bond, electron withdrawing groups in the compound exhibit this difference. Preferably the polar compound phase separates from a nonpolar liquid, is not soluble in a nonpolar liquid, or in an emulsion or suspension containing a nonpolar liquid and a polar liquid preferentially migrates to the polar phase. A nonpolar compound is a compound that has all of its covalent bonds having a similar electronic charge between the atoms bonded together, such compounds do not contain electron withdrawing groups. Nonpolar liquids preferably phase separate from a polar liquid or are not soluble in a polar liquid. Highly polar liquids phase separate from nonpolar liquids. Phase separation of polar and nonpolar liquids is based on the relative polarity of a pair of polar and nonpolar liquids. For the purpose of this invention nonpolar and highly polar liquids mean that a particular pair of liquids phase separate, wherein the liquid with the highest polarity is deemed highly polar and the liquid with the lower polarity is deemed nonpolar. Preferably a pair of liquids are considered to be an incompatible pair where the solubility of each in the other is about 5 percent by weight or less and more preferably about 1 percent by weight or less, based on the weight of the material dissolved in the major component. The difference in polarity of the two liquids is chosen such that they are insoluble in one another. As used herein polar active material or polar polymerizable component refers to a compound that is insoluble in the nonpolar phase or preferentially migrates to the highly polar liquid phase when a nonpolar liquid and a highly polar liquid are contacted. Some polar active materials may be soluble at some level in a nonpolar liquid but when the nonpolar liquid is contacted with a highly polar liquid, the active materials will migrate to and preferentially locate in the highly polar phase. Insoluble as used herein means that a material is not soluble in a particular liquid, preferably this means the material is soluble in an amount of about 5 percent by weight or less and most preferably about 1 percent by weight or less, based on the weight of the material dissolved in the particular liquid. Active material means herein a chemical species adapted to react with another chemical species or to perform a designated function once released from the capsule. Polymerizable component is a reactive compound that participates in the formation of a polymer once it is contacted with another polymerizable component, exposed to polymerization conditions or is a component that when exposed to certain conditions forms a polymer, such as a shell about the droplets of highly polar liquid contained in an emulsion or suspension with a nonpolar liquid. One or more as used herein means that at least one, or more than one, of the recited components may be used as disclosed. Nominal as used with respect, to functionality means the theoretical functionality, generally this can be calculated from the stoichiometry of the ingredients used. Generally, the actual functionality is different due to imperfections in raw materials, incomplete conversion of the reactants and formation of by-products. Residual content of a component refers to the amount of the component present in free form or reacted with another material, such as an adduct as described herein or a prepolymer. The residual content of a component can be calculated from the ingredients utilized to prepare the component or composition. Alternatively, it can be determined utilizing known analytical techniques. Substantially as used with respect to the absence of a component, such as surfactant, means that 1 percent by weight or less of the recited component is present, and more preferably about 0.1 percent by weight or less is present.

The core of the capsules of the invention comprises one or more highly polar liquids. The core is in essence die droplets formed during the emulsification or suspension of the highly polar liquid in the nonpolar liquid. The highly polar liquid may be a polar solvent or dispersant, an active material, a polymerizable component, a stabilizing additive (polymeric or otherwise) or a mixture thereof. A highly polar liquid may perform one or more of the functions of an active material and polymerizable component. In some embodiments the highly polar liquid is a solvent or dispersant for one or more active materials and/or one or more polar polymerizable components. Preferably the active material is a curing agent for a prepolymer or resin, (such as an epoxy resin, polyurethane, polyurea, aminoplast, thiourea, a cyanoacrylate and the like) a pharmaceutically active agent, a biocide, an insecticide, a herbicide, a catalyst for a reaction, an absorbent, a dye, a colorant, a photoactive agent, a stabilizer, an accelerator, a fragrance, a reactive intermediate, cells (for example red blood cells and white blood cells). RNA, DNA, proteins, sugars and the like. In one preferred embodiment the active material is one of more curing agents for an epoxy resin, any known curing agents for epoxy resins that is sufficiently polar to be located in the highly polar liquid may be used herein. Preferably the active material is a curing agent for one or more polyisocyanates or cyanoacrylates. Exemplary highly polar liquids include liquids containing one or more active hydrogen atom containing groups, ethers, thioeihers, sulphoxides, oxiranes, anhydrides, esters, and the like. Preferred highly polar liquids include water, amines, polyamines, alcohols, glycol ethers, amino alcohols, amides, sulfur oxides and the like. Even more preferred, highly polar liquids are water, methanol, glycerol, ethylene glycol, dimethyl formamide, dimethyl sulfoxide and the like. The core may comprise a polymer. The polymer, may be formed from the polymer forming components and partition to the highly polar liquid phase. The polymer may form in the highly polar phase as a result of the selection of the polymer forming components. Some, or all, of the polymer may locate in the highly polar phase. The polymer when located in the core can be mixed with the highly polar liquid and optionally with the active material. The polymer may encapsulate such materials in the core.

The shell comprises particles. Preferably the shell of the capsule comprises a polymer containing particles. The capsules can have an average size, largest diameter, sufficient for the ultimate use of the capsules and which contains a sufficient amount of active material for the desired use. The size of the capsules can be engineered for a variety of uses by adjusting the particle size, particle amount, dispersion conditions and other techniques known to one skilled in the art. Preferably the size of the capsules is about 50 nanometers or greater, more preferably about 500 nanometers or greater and most preferably about 5,000 nanometers or greater. Preferably the size of the capsules is about 500,000 nanometers or less, more preferably 50,000 nanometers or less and most preferably about 10,000 nanometers or less. The shell is of sufficient thickness and modulus to provide the desired strength of the capsules and to provide the desired highly polar liquid and/or active agent transmission properties, that is prevent the active material and/or highly polar liquid from leaking out of the particles or achieve a desired release rate of the active materials or highly polar liquid. The shell may have a thickness sufficient to prevent passage of the highly polar liquid or the active material through the shell. The shell may have a thickness and low enough free volume sufficient to control the passage of the highly polar liquid or the active material through the shell at a desired rate, that is provide controlled release of the highly polar liquid or active material. Preferably the thickness of the shell is about 10 microns or less and more preferably about 1 micron or less.

The polymer can comprise any polymer that can be formed at the interface of the droplets of highly polar liquid and the nonpolar liquid after emulsification. The polymer can be based on any polymer that ears be formed for example from processes including interfacial polymerization, in-situ polymerization, precipitations of the polymer from the nonpolar phase, anionic polymerization and electrostatic deposition, such as by coacervation or layer-by layer deposition. In interfacial polymerization the polymer forming components preferably comprise a relatively non-polar polymer forming component located in the nonpolar phase and a polar polymer forming component in the polar phase. Preferably the polymers prepared by interfacial polymerization are condensation polymers, which are well known in the art. In a more preferred embodiment the polymers prepared by interfacial polymerization include polyurethanes, polyureas, polyurethane-ureas, polyesters, aminoplasts, thioureas, polyvinyl addition polymers, formaldehyde condensates and the like. Preferably the shell comprises one or more polyureas. Preferably the polyureas are the condensation product of a polar (hydrophilic) polyamine and a nonpolar polyisocyanate. These materials are described in more detail in the section describing the preparation of the capsule of the invention. In anionic polymerization one or more anionically polymerizable monomers may be used to form the polymer in an emulsion.

The shell contains particles. The particles can be any particles that stabilize the droplets of the highly polar liquid in the polar liquid and which impart the desired strength and barrier properties to the transmission of active material through the shells. Preferably the particles are solid. The particles can be inorganic, organic or have both an organic and an inorganic component. Exemplary inorganic particles include metal salts, metals, metal alloys, metal oxides, metal sulfides, synthetic and naturally occurring minerals, mixtures thereof, clays and the like. The shape and aspect ratio of the particles can be any shape or aspect ratio that provides the desired properties to the shells, including platy, acicular (needle-like), cubic or spherical particles. The synthetic and naturally occurring minerals, including synthetic and naturally occurring clays generally comprise a mixture of two or more metal salts, metals, metal alloys, metal oxides, metal sulfides and the like. Among preferred minerals are silicon based minerals, for example silicates, colloidal silica, clays, modified clays and the like. Exemplary metal based particles include salts, oxides and hydroxides of calcium, magnesium, iron, zinc, nickel, titanium, aluminum, silicon, barium and manganese. Preferred metal salts include magnesium hydroxide, magnesium, carbonate, magnesium oxide, calcium oxalate, calcium carbonate, barium carbonate, barium sulfate, titanium dioxide, aluminum oxide, aluminum hydroxide and zinc sulfide. Exemplary minerals include silicates, bentonite, hydroxyapatite, alumina silicates, laponite, montmorilonite and hydrotalcites. The particles may comprise organic particles such as polymer particles. Any polymer particles of appropriate size which improve the strength of the shell and/or the active material and/or highly polar liquid barrier properties may be utilized. Exemplary polymer particles include crosslinked latex, polystyrene, hydrophobically modified cellulosic polymers, fluorinated polyolefins and fluorinated polyvinylidene particles, and the like. Exemplary hydrophobically modified cellulosic include acetylated nanofibers of cellulose polymers, silitated microfibrils of cellulosic polymers, carboxymethylated cellulose polymers and the like. The particles may comprise organic polymers containing metal, metal salts and the like. The particles may comprise inorganic particles modified with organic materials to improve the properties of the particle. The particles may comprise a mineral, for example a nanoclay, which is modified with an organic compound. An example of such modified inorganic particles includes nanoclays modified on their surfaces with an onium compound having at least one ligand with a hydrophobic group. Oniums are positively charged salts of nitrogen, phosphorous, sulfur and the like. A hydrophobic group is generally a long chain hydrocarbon group, preferably 5 carbons or greater and most preferably 8 carbons or greater. Preferred oniums are quaternary ammonium salts. Among preferred onium modified nanoclays are montmorillonite and fluoromica organo-clays modified with one or more ammonium chlorides containing a hydrophobic group, which are commercially available from Southern Clay products under the tradenames and designations of CLOISITE 20A, CLOISITE 30B, CLOISITE 10A and CLOISITE 93A nanoclays.

The particles may comprise electrically conductive particles and/or thermally conductive particles. The use of such particles can result in the production of networks of solid particles that are electrically and/or thermally conductive and can serve as a pathway to conduct or dissipate heat or electrical charge. Examples of electrically conductive particles include: particles of certain metal oxides, such as tin oxide, antimony-doped tin oxide, fluorine-doped tin oxide, indium-doped tin oxide, phosphorous-doped tin oxide, zinc antimonite, indium-doped zinc oxide, ruthenium oxide, rhenium oxide, silver oxide, nickel oxide, copper oxide, and the like; particles of carbon black, graphite, graphene, copper, silver, gold, nickel, tantalum, chromium, zirconium, vanadium, and niobium; as well as non-conductive particles, such as titanium dioxide, surface coated with an electrically conductive material, such as a tin oxide; and including mixtures of any of the foregoing particles. Examples of thermally conductive particles include: particles comprising aluminum oxide, aluminum nitride, boron nitride, boron carbide, silicon, carbide, silicon nitride, silicon oxide, magnesium oxide, magnesium nitride, titanium dioxide, zinc oxide, silver, gold, copper, carbon (including diamond) and metal coated materials, such as silver coated copper or sliver coated aluminum, as well as mixtures thereof.

The particles, such as silica and/or alumina particles, may be introduced into the emulsion in the form of colloidal dispersions, wherein finely divided solid particles are dispersed within a continuous medium in a manner that prevents them from being filtered easily or settled rapidly. Such dispersions are commercially available and an example is SNOWTEX-O, which is an aqueous colloidal silica sol having a pH of 2-4 and believed to contain 20 to 21 percent by weight nanosized (10-20 nanometers) silica particles dispersed in water.

The particles are generally of a size such that the desired properties of the capsules are achieved. The mean particle size of the particles is chosen to provide stable capsules, the desired strength and barrier properties. The mean particle size of the particles used is preferably about 3000 nm or less and more preferably about 1000 nm or less. The mean particle size of the particles used is preferably about 10 nm or greater, more preferably about 50 nm or greater and most preferably about 75 nanometer or greater. Preferably the particle size is from about 10 nm to 100 nm, particularly preferably from about 50 nm to 500 nm and most preferably from about 75 nm to 300 nm, in each case measured as the mean hydrodynamic equivalent diameter by means of photon correlation spectroscopy at 173° backscattering using a nanosizer ZS from Malver.

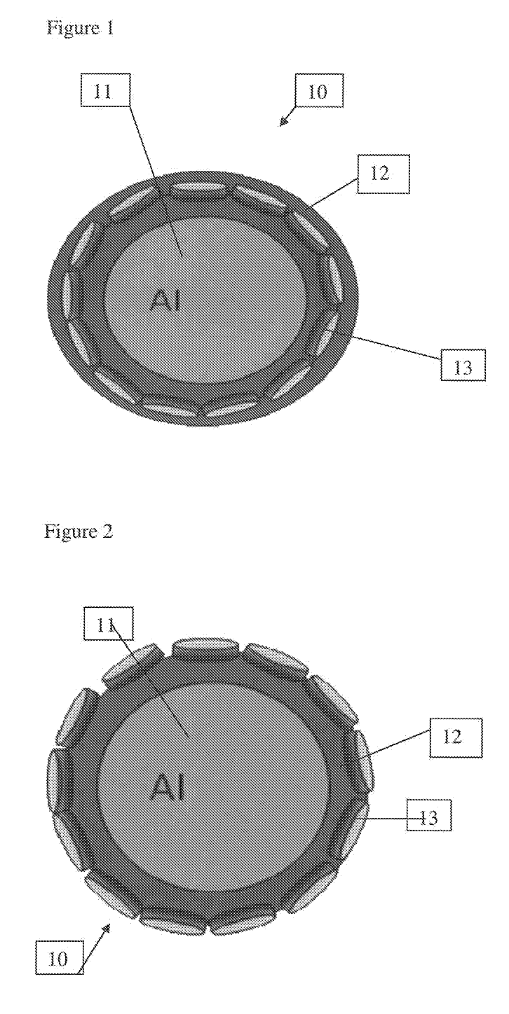

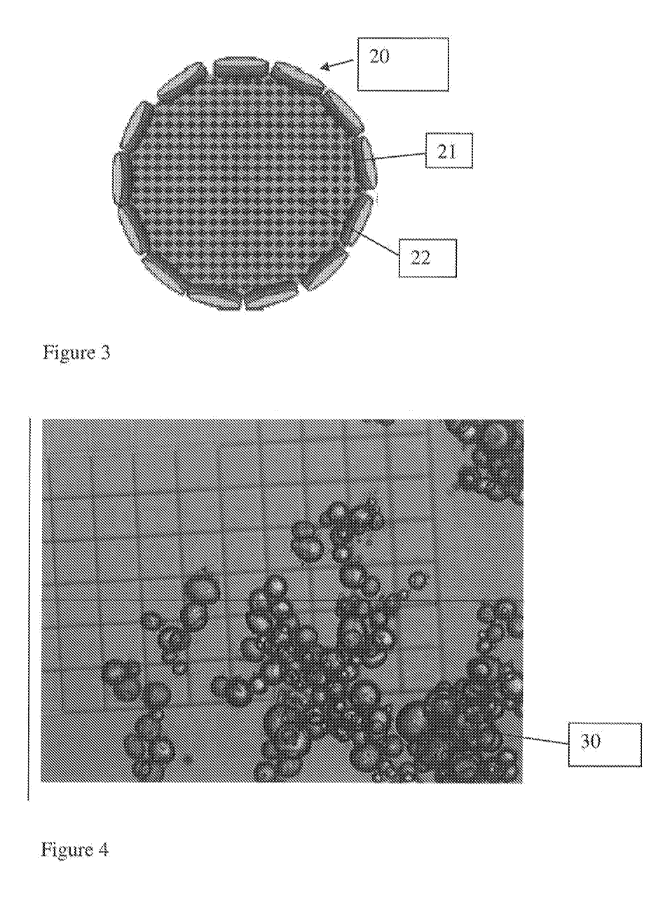

The particles may be partially or fully encapsulated in the polymer. Preferably the particles are uniformly distributed throughout the polymer shell. FIG. 1 shows one embodiment of the capsules of the invention. Shown is a capsule 10 which comprises a core of active material 11, a polymer shell 12 and particles 13 wherein the particles 13 are encapsulated in the shell 12. FIG. 2 shows a second embodiment of a capsule 10 which shows a core of active material 11 having a polymeric shell 12 about the core and particles 13 partially encased in the polymer shell 12. FIG. 3 shows a capsule 20 having a shell 21 of particles and a core 22 of an interpenetrating network of a polymer and active material.

The capsules may contain any other materials that are present in the emulsion or dispersion during capsule formation which materials do not impact the active materials or the function of the capsules, such as emulsifiers, surfactants, stabilizers and the like. The capsules may be prepared by the process comprising; a) contacting a dispersion of particles in one or more non-polar liquids with one or more immiscible highly polar liquids wherein the particles have a surface energy that promotes migration to the interface of the dispersion of the highly polar liquids in die nonpolar liquid continuous phase; b) emulsifying the contacted liquids to form an emulsion or suspension of the highly polar liquids in the non-polar liquids wherein discrete droplets of the highly polar liquids are formed having a portion of the particles on the surface of the droplets of the highly polar liquids; and c) forming a polymer, preferably a polymeric shells about the droplets of highly polar liquid wherein the polymeric shells comprise a portion of the particles. Preferably the particles are capable of stabilizing the droplets in the emulsion or suspension. Preferably the process is performed without the need for a surfactant. The particles are dispersed in one or more nonpolar liquids. Any nonpolar liquid that phase separates from the highly polar liquid may be used. Among preferred classes of non-polar liquids are aromatic hydrocarbons, aliphatic hydrocarbons, and she like. The concentration of particles is chosen to provide a sufficient amount of particles to provide the desired size of the capsules and the desired properties of the shell of the capsule. Higher concentrations of particles prepare smaller capsules and vice versa. Preferably the concentration of particles in nonpolar liquid is about 0.1 percent by weight or greater and more preferably about 1.0 percent by weight or greater based on the weight of the nonpolar liquid and the particles. Preferably the concentration of particles in nonpolar liquid is about 5.0 percent by weight or less and more preferably about 2.0 percent by weight or less based on the weight of the nonpolar liquid and the particles. The particles may be dispersed in the nonpolar solvent with agitation. Any known form of agitation may be utilized, such as ultrasonication, high-shear mixing, and the like. The nonpolar liquid may further comprise one or more polymeric stabilizers that do not prevent encapsulation, examples include polyisobutylene, polystyrene, any soluble polymer and the like. In embodiments wherein, the polymer is derived from nonpolar polymerizable components, such components may be dispersed in or dissolved in the nonpolar liquid prior to contacting it with the highly polar liquid. The active material and/or the polar polymerizable component are dissolved, suspended or dispersed in the one or more highly polar liquids. This can be achieved using standard techniques for dissolving or dispersing components in a liquid. Preferably this is achieved using known means of agitation.

The nonpolar liquids and the highly polar liquids are contacted and exposed to conditions such that an emulsion or suspension is prepared. The nonpolar liquids form the continuous phase and the highly polar liquids form the discontinuous phase. This is known as an inverse emulsion or suspension. The contacted liquids are subjected to one or more forms of agitation and/or shear to form the desired emulsion or suspension. Agitation and shear can be introduced through the use of impellers, ultrasonication, rotor-stator mixers and the like. For the industrial-scale production of emulsions or suspensions it is advisable to pass the mixture of nonpolar and highly polar liquids a number of times through a shear field located outside a reservoir/polymerization vessel until the desired droplet size has been reached. Exemplary apparati for generating a shear field are communication machines which operate according to the rotor-stator principle, e.g. toothed ring dispersion machines, colloid mills and corundum disk mills and high-pressure and ultrasound homogenizers. To regulate the droplet size, it can be advantageous to additionally install pumps and/or flow restrictors in the circuit around which the emulsion or suspension circulates.

Once a stable emulsion or suspension is formed the emulsion or suspension is subjected to polymerization conditions so as to form a polymer, preferably a polymer shell about the droplets of highly polar liquid. The conditions for polymerization, are based on the choice of the polymer utilized. Any polymer system and associated process for preparation may be used which forms a polymer or deposits or forms the polymer as a shell about the droplets. Exemplary processes include interfacial polymerization, in-situ polymerization, anionic polymerization, precipitation of the polymer from the polar or nonpolar phase and electrostatic deposition, such as by coacervation or layer-by layer deposition. Exemplary starting materials for such polymers include: in the case of using a coacervation method, anionic substances (e.g. gum arable, sodium alginate, copolymers of styrene-maleic anhydride, copolymers of vinyl methyl ether-maleic anhydride, phthalate esters of starch, and poly(acrylic acid)); in the case of anionic polymerization, cyanoacrylates, alkene substituted aromatics and alkadienes and the like; in the case of using an In-situ polymerization method, urea-formaldehyde resins, melamine-formaldehyde resins (melamine-formaldehyde prepolymers) and radically polymerizable monomers; and, in the case of using an interfacial polymerization method, preferably condensation polymers such as, combinations of hydrophilic monomers (e.g. polyamines, glycols, and polyphenols) and hydrophobic monomers (e.g. polybasic acid halides, bishaloformate, and polyisocyanates), from which capsule shells of such as polyamides, epoxy resins, polyurethanes, polyurea-urethanes and polyureas are formed.

The polymer may be formed by interfacial polymerization. Typically in interfacial polymerization a polar (or hydrophilic) polymer forming component is located in the highly polar liquid phase and a non-polar (hydrophobic) polymer forming component is located in the non-polar liquid. Other components that impact or enhance the polymerization can be added to one or the other of the highly polar liquid or nonpolar liquid based on the relative polarity (hydrophilicity or hydrophobicity) of the ingredient, examples of such additives include catalysts, accelerators, initiators, fillers, crosslinking agents, chain extenders, gelling agents, and the like. The polymerization is initiated by exposing the emulsion or suspension to conditions at which polymerization proceeds. Examples of this include adding ingredients, catalysts, initiators, accelerators, and the like; exposing the emulsion or suspension to temperatures at which polymerization proceeds at a reasonable rate; and the like. Such temperatures can be sub-ambent, ambient or super-ambient. In the embodiment wherein the polymerization proceeds at room temperature, such as for some reactions of polyisocyanates with compounds containing more than one active hydrogen containing groups, one of the ingredients is preferably added alter emulsification, in this embodiment it is preferable to add the nonpolar (hydrophobic) component after a stable emulsion or suspension is formed. This is because the continuous phase is nonpolar. Generally interfacial polymerization stops when the polymerizable components can no longer contact each other. In some embodiments, this occurs when the polymer shell effectively forms a barrier around the droplets.

The polymers prepared by interfacial polymerization preferably include polyureas, polyurethanes and polyurea-urethanes, which are generally prepared from polyisocyanates and compounds containing more than one isocyanate reactive compound. The polyisocyanates are generally nonpolar and dissolve or disperse in the nonpolar solvent. Polyisocyanates as used herein mean any polyisocyanate having more than one isocyanate group per molecule and preferably two or more isocyanate groups per molecule. Preferably the polyisocyanates have 4 or less isocyanate groups per molecule and more preferably 3 or less isocyanate groups per molecule. This preference assumes perfect reaction and ignores byproduct formation and is based on theoretical numbers of isocyanate groups that can be derived from the stoichiometry of the formation of such compounds. The polyisocyanates can be in the form of monomers, oligomers or prepolymers prepared from such monomers. The polyisocyanates for use in preparing the prepolymer include any aliphatic, cycloaliphatic, araliphatic, heterocyclic or aromatic polyisocyanates, or mixtures thereof. Preferably, the polyisocyanates used have an average isocyanate functionality of about 2.0 or greater and an equivalent weight of about 80 or greater. Preferably, the isocyanate functionality of the polyisocyanate is about 2.4 or greater; and is preferably about 4.0 or less. Higher functionality may also be used. Preferably, the equivalent weight of the polyisocyanate is about 110 or greater; and is preferably about 300 or less. Examples of preferable polyisocyanates include those disclosed by Wu. U.S. Pat. No. 6,512,033 at column 3, line 3 to line 49, incorporated herein by reference. More preferred isocyanates are aromatic isocyanates, alicyclic isocyanates and derivatives thereof. Preferably, the aromatic isocyanates have the isocyanate groups bonded directly to aromatic rings. Even more preferred polyisocyanates include diphenylmethane diisocyanate and oligomeric or polymeric derivatives thereof, isophorone diisocyanate, tetramethylxylene diisocyanate, 1,6-hexamethylene diisocyanate and polymeric derivatives thereof, bis(4-isocyanatocylohexyl)methane, and trimethyl hexamethylene diisocyanate. Most preferred isocyanates include diphenylmethane diisocyanate and oligomeric or polymeric derivatives thereof. The amount of isocyanate containing compound used to prepare the prepolymer is that amount that gives the desired properties, such as the desired shell thickness and morphology. Preferably the isocyanate functional prepolymers are the reaction product of one or more polyisocyanates and one or more isocyanate reactive compounds wherein an excess of polyisocyanate is present on an equivalents basis.

The other polymerizable component reacted with the polyisocyanates are isocyanate reactive compounds. Preferably these are polar polymerizable components, that is, they preferentially dissolve or disperse in the highly polar liquid. The term isocyanate-reactive compound with respect to the polar polymerizable components as used herein includes any organic compound having normally at least two isocyanate-reactive moieties. For the purposes of this invention, an isocyanate reactive moiety, active hydrogen containing moiety, refers to a moiety containing a hydrogen atom which, because of its position in the molecule, displays significant activity according to the Zerewitinoff test described By Wohler in the Journal of the American Chemical Society Vol. 49, p. 3181 (1927). Illustrative of such active hydrogen moieties are —COOH, —OH, —NH₂, —N—, —CONH₂—, —SH, and —CONH—. Preferable isocyanate reactive compounds, polar polymerizable components, include water, polyols, polyamines, polymercaptans and polyacids. More preferably, the isocyanate reactive compound is one or more polyamines. Preferably the one or more polyamines comprise the highly polar liquid or preferentially partitions in the highly polar liquid. Exemplary polyamines include: aliphatic amines, such as ethylenediamine, diethylenetriamine, triethylenetetramime, tetraethylenepentamine, 1,3-propylenediamine, and hexamethylenediamine; epoxy compound addition products from aliphatic polyamines, such as poly(C₁₋₅)alkylene(C₁₋₆)polyamine-alkylene (C₂₋₁₈) oxide addition products; aromatic polyamines, such as phenylenediamine, diaminonaphthalene, and xylylenediamine; alicyclic polyamines such as piperazine; and heterocyclic diamines such as 3,9-bis-aminopropyl-2,4,8,10-tetraoxaspiro-[5,5]undecane. Among preferred polyamines are polyethyleneimine, tetraethylenepentamine, diethylenetriamine, 2-aminoetylethanolamime, ethylene diamine, triethylene tetramine, piperazine, aminoethyl piperazine, and the like. Preferably the polyamine does not contain a hydrophobic group, for instance a cycloaliphatic group, an aromatic group or a carbon chain, of 6 carbons or greater which hydrophobic group does not contain an electron withdrawing group. Known catalysts, initiators, gelling agents, crosslinking agents or chain extenders may be included in either the nonpolar phase or the highly polar phase.

In another embodiment, the polymer may be formed by in-situ polymerization. Any in situ polymerization technique may be used which is capable of forming a polymer and preferably a polymeric shell around the droplets of highly polar liquid dispersed in the nonpolar liquid. In a preferred embodiment die polymer shell is formed from one or more free radically polymerizable monomers. Preferably the monomers contain olefinic unsaturation. The monomers and initiators and/or photocatalysts are dispersed or dissolved in the nonpolar phase. Once a stable emulsion or suspension is prepared, the emulsion or suspension is exposed to conditions such that polymerization proceeds. The emulsion or suspension is exposed to conditions such that free radical formation occurs such that polymerization proceeds. The emulsion or suspension can be exposed to temperatures or to ultraviolet light such that free radicals are formed and polymerization proceeds. Alternatively an initiator cats be added to the emulsion or suspension which initiates free radical formation. Any other known means for initiating free radical polymerization may be utilized. Exemplary monomers and conditions useful for in-situ polymerization are described in U.S. Pat. No. 7,572,397 incorporated herein by reference.

Where the polymer is located in the core, this is achieved by choosing a polymer that is highly polar and partitions to the highly polar phase or by selecting components that form the polymer in the highly polar phase.

Formation of a polymer shell by coacervation is known to those skilled in the art, one example is described in U.S. Pat. No. 3,539,465, incorporated herein by reference. Formation of a polymer shell by layer-by-layer deposition, such as by deposition precipitation, is known in the art. The polymer prepared by anionic polymerization may be any polymer that can be formed by anionic polymerization. Classes of monomers that may be used to prepare anionic polymers include cyanoacrylates, alkadienes, alkene substituted aromatic compounds and mixtures thereof. Polymers of cyanoacrylates and copolymers of cyanoacrylates with other monomers polymerizable by anionic polymerization are preferred. Preferred alkadienes are conjugated dienes, such, as butadiene and isoprene. Preferred alkenyl aromatics include styrene and substituted versions thereof. Preferred classes of cyanoacrylates include alkyl, alkoxyalkyl and alkenyl cyanoacrylates. Preferred are C₁-C₁₀ alkyl, (C₁-C₄)alkoxy(C₁-C₁₀)alkyl; or C₂-C₁₀ alkenyl cyanoacrylates. Exemplary cyanoacrylates include ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-oetyl 2-cyanoacrylate, ally 2-cyanoacrylate, methoxypropyl 2-cyanoacrylate and isoamyl cyanoacrylate. The anionic polymers may be prepared by contacting a highly polar (water) phase, optionally containing one or more surfactants, preferably no surfactants, with a hydrophobic phase containing a nonpolar solvent and the anionic polymers dissolved or dispersed therein. The hydrophobic phase may contain a nucleophilic agent to initiate polymerization as disclosed in US 2007/025930 paragraph 0198 to 0200, incorporated herein by reference. The pH of the water phase is preferably adjusted with acids, bases or buffers, such as phosphate buffers and buffers available from FisherScientific. Preferably the pH is adjusted to about 4 to 10, and most preferably about 7 to 8. Solvents and surfactants useful in this process are disclosed in US 2007/025930 paragraphs 0012 to 0022 and 0040 to 0043 incorporated herein by reference. Where copolymers are prepared by anionic polymerization preferably 50 percent by weight or greater of the monomers are cyanoacrylate and more preferably 70 percent by weight or greater. Once the solutions and or dispersions are contacted the reaction proceeds. Generally the reaction proceeds at room temperature but higher or lower temperatures maybe utilized to adjust the rate of polymerization.

After the polymer shells are formed on the droplets the capsules may be recovered by any known technique that does not substantially harm the capsules. Exemplary processes for recovery of the capsules include filtration of the capsules from the continuous phase, precipitation, spray drying, decantation, centrifugation, flash drying, freeze drying, evaporation, distillation and the like. The separation process is selected to effect a rapid and efficient separation, with a minimum of mechanical damage to or disruption of the microcapsules.

### Illustrative Embodiments of the Invention

The following examples are provided to illustrate the invention, but are not intended to limit the scope thereof. All parts and percentages are by weight unless otherwise indicated.

### EXAMPLES 1 TO 23

Capsule Preparation Procedure

To a 120 mL (milliliters) flat bottom jar with magnetic stir bar add 1.3 pph (part per hundred) of polyisobutylene (Weight average molecular weight 500,000 Daltons) in xylenes (30 g), 5 pph quaternary amine modified, nanoclay in xylenes (0.6 g), and a premixed solution of diethylene triamine (3 g) and water (6 g). Thoroughly mix and then ultrasonicate (Sonics VCX 500 Watt model) at 50 percent power (4×5 seconds). Stir the prepared emulsion at 1500 rpm for 2 minutes. Add a solution of 0.5 g polymeric methylene diisocyanate in 5.5 g xylenes using a large pipette. This addition is done quickly (<3 sec) and smoothly with rapid stirring maintained throughout. Then reduce the stirring speed to 500 rpm. After 10 minutes, dilute with fresh xylenes (30 mL) and stop stirring. When capsules settle, the supernatant may be tested for isocyanate peak (˜2275 cm⁻¹) by ATR-IR. This peak is stable overnight, indicating a barrier has been formed between the isocyanate and the amine. To work up capsules, the sample is repeatedly decanted and treated with fresh xylenes until no isocyanate peak is visible by ATR-IR. FIG. 4 shows an optical micrograph of capsules 30 formed. The nanoclay suspension is prepared as 5 pph suspension in xylenes. Suspension, is prepared by slow addition of nanoclay powder to stirring xylenes followed by bath sonication for 1 hour with brief stirring every five minutes. The stock suspension is then ultrasonicated (3×5 sec at 50% power) to maximize exfoliation.

A number of other polar polar active materials are utilized to prepare capsules of the invention. These are listed in Table 1. The active materials in these examples are encapsulated in polyurea shells using polymeric methylenediphenyl diisocyanate MDI having on average 2.7 equivalents per mole available from The Dow Chemical Company, Midland Mich. under the trademark PAPI™ 27. A number of particles are used in preparing capsules of the invention which are listed in Table 2.

TABLE 1
Polar Active Materials

| Name | Acronym | Structure |
|---|---|---|
| polyethyleneimine | PEI | 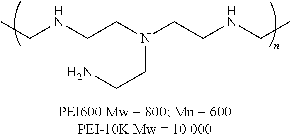 |
| tetraethylenepentamine | TEPA | 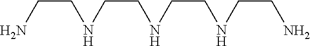 [SMILES: NCCNCCNCCNCCN] |
| diethylenetriamine | DETA | 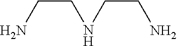 |
| 2- aminoethylethanolamine | AEEA | 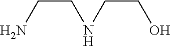 |
| L ascorbic acid | Vitamin C | 3,4-dihydroxy-S-((S)-1.2dihydroxyethyl) furan-2-one |
| Laccase M120 | Polyphenol | Polyphenol oxidase, an amino acid sequence forming |
| | oxidase | an enzyme having CAS #80498-15-3 |

TABLE 2
Particles Used

| Particle | Designation | Description |
|---|---|---|
| Closite 20A | 1 | Quaternary amine modified clay, see below |
| Closite 30B | 2 | Quaternary amine modified clay, see below |
| Closite 10A | 3 | Quaternary amine modified clay, see below |
| Closite 93A | 4 | Quaternary amine modified clay, see below |
| Polytetrflouroethyelene particles | 5 | 1 microm particles |
| Polyflourovinylidene particles | 6 | Mw 534,000 |

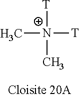

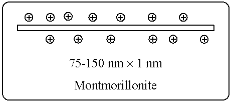

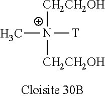

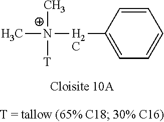

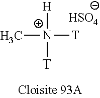

Several, capsules are prepared using the procedure described above and the ingredients listed in Table 3

TABLE 3

| | | | | Polyiso | Nonpolar | | |
|---|---|---|---|---|---|---|---|
| | | Water | Particle | butylene | solvent | Isocyanate | |
| Ex | Polar Active (g) | (g) | Designation | (g) | Xylene (g) | (g) | Notes |
| 1 | DETA 3 g | 6 | 2 (0.03) | 0.39 | 30 | 6 | |
| 2 | none | 6 | 1 (0.03) | 0.39 | 30 | 1.11 | 1, 2, 5 |
| 3 | PE1600 1.91 g | 6 | 1 (0.03) | 0.39 | 30 | 1.11 | 1 |
| 4 | PE1600 1.91 g TEPA 1.33 g | 6 | 1 (0.03) | 0.39 | 30 | 1.11 | 1, 2 |
| 5 | PE1600 1.91 g TEPA 0.67 g | 6 | 1 (0.03) | 0.39 | 30 | 1.11 | 1 |
| | AEEA 0.91 | | | | | | |
| 6 | TEPA 1.33 g AEEA 1.82 g | 6 | 1 (0.03) | 0.39 | 30 | 1.11 | 1 |
| 7 | TEPA 1.33 | 6 | 1 (0.03) | 0.39 | 30 | 1.11 | 1, 2 |
| 8 | DETA 2 | 2 | 1 (0.03) | 0.39 | 30 | 1.11 | 1, 2 |
| 9 | DETA 4 | 4 | 1 (0.03) | 0.39 | 30 | 1.11 | 1, 2 |
| 10 | DETA 6 | 6 | 1 (0.03) | 0.39 | 30 | 1.11 | 1, 2, 3 |
| 11 | DETA 2 | 6 | 1 (0.03) | 0.39 | 30 | 1.11 | 1, 2 |
| 12 | DETA 2 | 6 | 4 (0.03) | 0.39 | 30 | 1.0 | 1 |
| 13 | DETA 2 | 6 | 4 (0.03) | 0.39 | 30 | 0.5 | 1, 2 |
| 14 | DETA 2 | 6 | 2 (0.03) | 0.39 | 30 | 0.5 | 1, 2 |
| 15 | DETA 2 | 6 | 1 (0.03) | 0.39 | 30 | 0.5 | 1 |
| 16 | DETA 2 | 6 | 2 (0.03) | 0.39 | 30 | 1.0 | 1 |
| 17 | DETA 2 | 6 | 1 (0.03) | 0.39 | 30 | 1.0 | 1 |
| 18 | PE1600 1.37 g | 6 | 1 (0.01) | 0.39 | 30 | 1.0 | 1, 2 |
| 19 | PE1600 1.91 g | 6 | 1 (0.01) | 0.78 | 30 | 1.0 | 1, 2 |
| 20 | TEPA 4 | 6 | 5 (0.2) | 0.39 | 35.1 | 0.2 | |
| | | | 6 (0.25) | | | | |
| 21 | Polyphenol oxidase 0.3 g | 6 | 2 (0.024) | 0.39 | 35.1 | 0.2 | |
| 22 | Vitamin C 0.54 g | 6 | 2 (0.024) | 0.39 | 35.1 | 1.2 | |

Isocyanate is provided as 0.5 g in 5.5 g of xylene in Example 1. In Examples 20 and 21 isocyanate is provided as 0.2 g in 5 g of xylene by inverting a small vial of the solution over the reaction mixture. In Example 22 isocyanate is provided as 1.2 g in 5 g of xylene by inverting a small vial of the solution over the reaction mixture.

### EXAMPLES 23-28

### Cyanoacrylate Polymer

Capsules of the invention are prepared from cyanoacrylate monomers using the ingredients listed in Table 4 and the procedure listed below.

TABLE 4

| Ingredients | | | | | | |
|---|---|---|---|---|---|---|
| in order of addition | 23 | 24 | 25 | 26 | 27 | 28 |
| Xylene (g) | 30 | — | — | 30 | 301 | |
| Heptane (g) | — | 30 | 30 | — | — | 30 |
| Fisher Buffer pH 4 (g) | 6 | — | 6 | — | — | — |
| Fisher Buffer pH 7 (g) | — | — | — | 6 | — | 6 |
| Fisher Buffer pH 10 (g) | | 6 | — | — | 6 | — |
| 5 pph Particle 1 in xylene | 0.5 | — | — | 0.5 | 0.5 | — |
| (g) | | | | | | |
| 5 pph Particle 1 in heptane | — | 0.5 | 0.5 | — | — | 0.5 |
| (g) | | | | | | |
| Cyanoethylacrylate (g) | 0.3 | 0.3 | 0.3 | 0.3 | 0.3 | 0.3 |

The solvent is weighed into a 60 milliter jar and a stir bar is added. The buffer in water is added and the stir bar is stirred at 500 rpm to dissolve or disperse the buffer. The particles are added and the mixture is ultrasonicated at 50 percent power (4×5 sec and the jar is closed and shaken between cycles). The mixture is stirred at 500 rpm after sonication. The monomer is added. The mixtures are examined using optical microscopy and capsule morphologies are visible in all examples.

Parts by weight as used herein refers to 100 parts by weight of the composition specifically referred to. In most cases, this refers to the composition of this invention. The preferred embodiment of the present invention has been disclosed. A person of ordinary skill in the art would realize however, that certain modifications would come within the teachings of this invention. Therefore, the following claims should be studied to determine the true scope and content of the invention.

Any numerical values recited in the above application include all values from the lower value to the upper value in increments of one unit provided that there is a separation of at least 2 units between any lower value and any higher value. As an example, if it is stated that the amount of a component or a value of a process variable such as, for example, temperature, pressure, time and the like is, for example, from 1 to 90, preferably from 20 to 80, more preferably from 30 to 70, it is intended that values such as 15 to 85, 22 to 68, 43 to 51, 30 to 32 etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner. Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at feast the specified endpoints. Parts by weight as used herein refers to compositions containing 100 parts by weight. The disclosures of all articles and references, including patent applications and publications, are incorporated by reference for all purposes. The term “consisting essentially o” to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of the elements, ingredients, components or steps. Plural, elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps.

What is claimed is:

1. A composition comprising a plurality of capsules wherein the capsules comprise:
a core of one or more highly polar liquids; one or more polar active materials dissolved in or dispersed in one or more highly polar liquids; a mixture of one or more polymers and one or more highly polar liquids; or a mixture of one or more polymers, one or more highly polar liquids and one or more polar active materials, and
a shell comprising particles in a polymer matrix or particles;
wherein the thickness of the shell is sufficient to prevent passage of the highly polar liquid or the active material through the shell or to control the rate passage of the highly polar liquid or the active material through the shell with the proviso that the one or more polymers may be located in the core, in the shell or both.

2. A composition according to claim 1 wherein the core comprises one or more highly polar liquids or one or more polar active materials dissolved in or dispersed in one or more highly polar liquids; and the shell comprises particles in a polymer matrix.

3. A composition according to claim 1 wherein the core comprises; a mixture of one or more polymers and one or more highly polar liquids; or a mixture of one or more polymers, one or more highly polar liquids and one or more polar active materials; and the shell comprises particles.

4. A composition according to claims 1 wherein the capsules are substantially free of a surfactant.

5. A composition according to claim 1 wherein the polymer is formed by interfacial polymerization, coacervation, anionic polymerization or in situ polymerization.

6. A composition according to claim 1 wherein the polymer is formed by interfacial polymerization and is a polyurea, polyurethane, polyurea-urethane or a mixture thereof.

7. A composition according to claim 1 wherein the particles are solid particles that have a surface energy that promotes migration to the interface of the interface of an emulsion or suspension of a highly polar liquid in a nonpolar liquid.

8. A process comprising;
a) contacting a dispersion of particles in a non-polar liquid with a highly polar liquid wherein the particles have a surface energy that promotes migration to the interface of the emulsion or suspension of the highly polar liquid in the nonpolar liquid;
b) emulsifying the contacted liquids to form an emulsion or suspension of the highly polar liquid in the non-polar liquid wherein discrete droplets of the highly polar liquid are formed having a portion of the particles on the surface of the droplets of highly polar liquid; and,
c) forming a polymer which forms a polymeric shell about the droplets of highly polar liquid wherein the polymeric shells comprise a portion of the particles; forms a mixture of the polymer and the highly polar liquid, and optionally the active material, in the core; or both.

9. A process according to claim 8 wherein step c) comprises forming polymeric shells about the droplets of highly polar liquid wherein the polymeric shells comprise a portion of the particles.

10. A process according to claim 8 wherein the highly polar liquid is a polymer forming component.

11. A process according to claim 8 wherein the highly polar liquid contains one or more active materials, polymer forming components or a mixture thereof.

12. A process according to claim 8 wherein the highly polar liquid contains a polymer forming component and an active material.

13. A process according to claim 8 wherein the polymer is formed by interfacial polymerization and the polymer is prepared from a non-polar polymer forming component and a polar polymer forming component wherein the polar polymer forming component Is dissolved or dispersed in the highly polar liquid and the nonpolar polymer forming component is introduced through the non-polar component.

14. A process according to claim 8 wherein the nonpolar polymer forming component comprises one or more polyisocyanates and the polar polymer forming component comprises one or more components containing more than one isocyanate reactive groups.

15. A process according to claim 8 wherein the polymer is formed by in-situ polymerization and the nonpolar liquid contains one or more polymer forming components comprising free radically polymerizable monomers, oligomers or prepolymers and one or more free radical initiators.

16. A process according to claim 8 wherein the polymer is formed from monomers comprising cyanoacrylates.

17. A process according to Claim 8 wherein the polymer is formed by coacervation.

18. A process according to claim 8 wherein the process is performed in the substantial absence of a surfactant.

19. A process according to claim 8 wherein the particles are solid particles that have a surface energy that promotes migration to the interface of an emulsion or suspension of a highly polar liquid in a nonpolar liquid.

20. A composition according to claim 1 wherein the active material comprises a curing agent for a prepolymer or resin, a pharmaceutically active agent, a biocide, an insecticide, a herbicide, a catalyst for a reaction, an absorbent a dye, a colorant, a photoactive agent, a stabilizer, an accelerator, a fragrance, a reactive Intermediate, cells, RNA, DNA, a protein or a sugar.
